(19) United States
(12) Patent Application Publication
(10) Pub. No.: US 2026/0240931 A1
(43) Pub. Date: Aug. 20, 2026

(54) PHARMACEUTICAL COMPOSITION FOR PREVENTING OR TREATING HAIR LOSS,CONTAINING CONNARUS SEMIDECANDRUS JACK. EXTRACT

(71) Applicants: REPUBLIC OF KOREA (NATIONAL INSTITUTE OF BIOLOGICAL RESOURCES), Incheon (KR); PHARMACOBIO, INC., Seongnam-si (KR)

(72) Inventors: Jun Mee HUR, Goyang-si (KR); Sa Rah LEE, Goyang-si (KR); Hye Woo BYUN, Seoul (KR); Byoung Hee LEE, Seoul (KR); Omaliss KEO, Phnom Penh (KH); Masphal KRY, Phnom Penh (KH); Sung Min KANG, Yongin-si (KR); Yang Kook RHO, Seongnam-si (KR)

(21) Appl. No.: 18/997,501

(22) PCT Filed: Jun. 23, 2023
(86) PCT No.: PCT/KR2023/008732

§ 371 Date: Jan. 21, 2025

(30) Foreign Application Priority Data
Jul. 6, 2022 (KR) 10-2022-0083280

(51) Int. Cl. A61K 36/185; A61P 17/14

(52) U.S. Cl. CPC A61K 36/185; A61P 17/14

(57) ABSTRACT

The present disclosure relates to a composition for preventing, alleviating or treating hair loss and a composition for promoting hair growth or hair regrowth, both compositions containing a Connarus semidecandrus Jack. extract. According to the present disclosure, it has been identified that when a testosterone-induced alopecia animal model is treated with the Connarus semidecandrus Jack. extract, hair growth is promoted, hair length and thickness are also increased, the expression of androgen receptors and apoptosis-related factors is inhibited, the survival and proliferation of human dermal papilla cells are promoted, and the activity of 5-α reductase is inhibited, and thus the Connarus semidecandrus Jack. extract of the present disclosure can be effectively and variously used for preventing or treating hair loss and promoting hair growth or hair regrowth.

### CROSS-REFERENCE TO RELATED APPLICATIONS

This application is a National Stage of PCT/KR2023/008732, filed Jun. 23, 2023, which claims priority to Korean patent application no. 10-2022-0083280.

### TECHNICAL FIELD

The present disclosure relates to a Connarus semidecandrus jack. extract having effects of promoting hair growth or hair regrowth and preventing or treating hair loss.

### BACKGROUND ART

Hair repeats at cycles in the order of growth (anagen), involution (catagen), and rest (telogen) phases. The duration of the growth phase is 2 to 6 years and accounts for 85 to 90% of all hair follicles, and the duration of the involution phase is 2 weeks and accounts for 2 to 3% of all hair follicles. Finally, the duration of the rest phase is 3 to 4 months and accounts for 10 to 15% of all hair follicles. Such an overall cycle is repeated, but as the cycle gradually becomes shorter, and the hair that has fallen out at an average of 30 to 100 per day falls out more or stops growing, which is called alopecia.

Alopecia may be broadly classified into three types based on symptoms: congenital alopecia, in which hair is absent from birth; acquired alopecia, in which hair gradually falls out with age; and alopecia caused by pathological causes. Acquired alopecia includes the most common alopecia areata, male pattern alopecia (androgenetic alopecia) caused by genetic predisposition and androgen action on the hair, alopecia neurotica, pityriasis alopecia, alopecia seborrhecia, middle-aged alopecia, senile alopecia, and the like. Alopecia caused by pathological causes includes postpartum alopecia, postfebrile alopecia, drug-induced alopecia, and the like.

Currently, the most effective drugs for preventing or treating hair loss may include, for example, Minoxidil and Propecia. Minoxidil was originally an antihypertensive and a drug that was created to expand capillaries and lower blood pressure, but as Minoxidil is known to have the side effect of alleviating blood flow to the hair roots and thickening the fine hair to affect hair growth, Minoxidil has been used as a hair growth agent, unlike the original purpose. However, Minoxidil also affects the hair on the whole body, thereby causing the hair on the whole body besides hair on head to grow darker and thicker, and lowering blood pressure, and as a result, people with low blood pressure should refrain from using the drug, and because the drug is metabolized in the liver, people with liver disorders should also refrain from using the drug.

The ingredient name of Propecia is finasteride, which was developed by the Merck pharmaceutical company in USA. This ingredient was developed to treat benign prostatic hyperplasia, but during the research process, it was found that the ingredient could promote hair growth, and thus the finasteride was used as a hair loss therapeutic agent. When describing the cause of hair loss, testosterone is irreversibly metabolized into dihydrotestosterone (DHT) by 5α-reductase, and DHT binds to an androgen receptor (AR) to exhibit various biological effects. In human hair follicles, AR is mainly distributed in dermal papilla cells, and DHT binds to AR in dermal papilla cells to promote the secretion of hair growth inhibitory factors such as TGF-β (transforming growth factor-beta), and these inhibitory factors induce the death of hair matrix cells. As a result, the growth period of the hair follicles is gradually shortened and the hair follicles become miniaturized, which leads to hair loss. Propecia inhibits hair loss by inhibiting 5α-reductase, which causes hair loss to reduce the concentration of DHT. However, Propecia is a male hormone, which is prohibited for women, and is known to have side effects such as loss of libido and sexual dysfunction. Recently, depression and mood deformation have been reported in patients who took Propecia, which has been added as a precaution. Therefore, there is a need to develop a hair loss therapeutic agent that is more effective and has fewer side effects than Minoxidil and Propecia.

### BRIEF SUMMARY

### Technical Problem

The present inventors confirmed that a Connarus semidecandrus Jack. extract had effect of promoting hair growth or hair regrowth and preventing or treating hair loss while searching for a substance derived from a natural product that was relatively safe compared to chemical substances and had a hair loss treatment effect, and then completed the present disclosure.

An object of the present disclosure is to provide a pharmaceutical composition for preventing or treating hair loss.

Another object of the present disclosure is to provide a quasi-drug composition for preventing or alleviating hair loss.

Yet another object of the present disclosure is to provide a cosmetic composition for preventing or alleviating hair loss.

Still another object of the present disclosure is to provide a food composition for preventing or alleviating hair loss.

Still another object of the present disclosure is to provide a health functional food composition for preventing or alleviating hair loss.

Still another object of the present disclosure is to provide a pharmaceutical composition for promoting hair growth or hair regrowth.

Still another object of the present disclosure is to provide a quasi-drug composition for promoting hair growth or hair regrowth.

Still another object of the present disclosure is to provide a cosmetic composition for promoting hair growth or hair regrowth.

Still another object of the present disclosure is to provide a food composition for promoting hair growth or hair regrowth.

Still another object of the present disclosure is to provide a health functional food composition for promoting hair growth or hair regrowth.

Still another object of the present disclosure is to provide a method for treating hair loss.

Still another object of the present disclosure is to provide a method for promoting hair growth or hair regrowth.

### Technical Solution

An aspect of the present disclosure provides a pharmaceutical composition for preventing or treating hair loss containing a Connarus semidecandrus Jack. extract.

Another aspect of the present disclosure provides a quasi-drug composition for preventing or alleviating hair loss containing a Connarus semidecandrus Jack. extract.

Yet another aspect of the present disclosure provides a cosmetic composition for preventing or alleviating hair loss containing a Connarus semidecandrus Jack. extract.

Yet another aspect of the present disclosure provides a food composition for preventing or alleviating hair loss containing a Connarus semidecandrus Jack. extract.

Still another aspect of the present disclosure provides a health functional food composition for preventing or alleviating hair loss containing a Connarus semidecandrus Jack. extract.

Still another aspect of the present disclosure provides a pharmaceutical composition for promoting hair growth or hair regrowth containing a Connarus semidecandrus Jack. extract.

Still another aspect of the present disclosure provides a quasi-drug composition for promoting hair growth or hair regrowth containing a Connarus semidecandrus Jack. extract.

Still another aspect of the present disclosure provides a cosmetic composition for promoting hair growth or hair regrowth containing a Connarus semidecandrus Jack. extract.

Still another aspect of the present disclosure provides a food composition for promoting hair growth or hair regrowth containing a Connarus semidecandrus Jack. extract.

Still another aspect of the present disclosure provides a health functional food composition for promoting hair growth or hair regrowth containing a Connarus semidecandrus Jack. extract.

Still another aspect of the present disclosure provides a method for treating hair loss including treating a subject with a Connarus semidecandrus Jack. extract.

Still another aspect of the present disclosure provides a method for promoting hair growth or hair regrowth including treating a subject with a Connarus semidecandrus Jack. extract.

### Advantageous Effects

According to the present disclosure, it has been identified that when a testosterone-induced alopecia animal model was treated with the Connarus semidecandrus Jack. extract, hair growth was promoted, hair length and thickness were also increased, the expression of androgen receptors and apoptosis-related factors was inhibited, the survival and proliferation of human dermal papilla cells were promoted, and the activity of 5-α reductase was inhibited. Therefore, the Connarus semidecandrus Jack. extract of the present disclosure can be effectively and variously used for preventing or treating hair loss and promoting hair growth or hair regrowth.

### BRIEF DESCRIPTION OF DRAWINGS

FIG. 1 is a diagram illustrating an experimental timeline for confirming hair growth promotion and hair thickening effects of a Connarus semidecandrus Jack. extract in an androgenetic alopecia animal model.

FIG. 2 is a diagram illustrating a result of confirming hair growth promotion and hair thickening effects of a Connarus semidecandrus Jack. extract according to the present disclosure in an androgenetic alopecia animal model.

FIG. 3A shows a result of scoring a skin color of each experimental group based on the result of FIG. 2 above, and FIG. 3B shows a result of showing a hair type frequency of each experimental group based on the result of FIG. 2 above. In FIG. 3B, # indicates a value of p<0.05 compared to a normal group (Normal), ## indicates a value of p<0.01 compared to the normal group, * indicates a value of p<0.05 compared to a vehicle experimental group (testosterone-treated experimental group), and ** indicates a value of p<0.01 compared to the vehicle experimental group.

FIG. 4A is a result showing a photograph of a hair type of each experimental group based on the result of FIG. 2 above, FIG. 4B is a result showing a thickness of each hair type in each experimental group based on the result of FIG. 2 above, and FIG. 4C is a result showing a total hair thickness of each experimental group based on the result of FIG. 2 above.

FIG. 5A shows a histological analysis result of hair follicles in an androgenetic alopecia animal model treated with a Connarus semidecandrus Jack. extract according to the present disclosure, FIG. 5B shows a result of calculating a ratio of decomposed hair fibers (black arrows in FIG. 5A) among the total hair fibers of each experimental group, FIG. 5C shows images of animal model hair follicles cultured in vitro for 0 hour, 8 hours, 16 hours, 24 hours, or 48 hours using dimethyl sulfoxide (DMSO) or a Connarus semidecandrus Jack. extract according to the present disclosure, and FIG. 5D shows a result of the lengths of animal model hair follicles cultured in vitro for 0 hour, 8 hours, 16 hours, 24 hours, or 48 hours using a Connarus semidecandrus Jack. extract according to the present disclosure based on FIG. 5C. In FIG. 5A above, the black arrows indicate decomposed hair fibers (empty spaces).

FIG. 6 shows a result of confirming an effect of a Connarus semidecandrus Jack. extract according to the present disclosure on the expression of an androgen receptor (AR) protein increased by testosterone treatment.

FIG. 7A shows a result of confirming an effect of a Connarus semidecandrus Jack. extract according to the present disclosure on the expression of Bcl-2 mRNA decreased by testosterone treatment, FIG. 7B shows a result of confirming an effect on the expression of Bax mRNA increased by testosterone treatment, and FIG. 7C shows a result of confirming an effect on the expression of Caspase-3 (Caspase-9) mRNA increased by testosterone treatment. In FIG. 7, ## indicates a value of p<0.01 compared to a normal group, * indicates a value of p<0.05 compared to an untreated experimental group of a Connarus semidecandrus Jack. extract according to the present disclosure, and ** indicates a value of p<0.01 compared to the untreated experimental group of a Connarus semidecandrus Jack. extract according to the present disclosure.

FIG. 8 shows a result of confirming an effect of a Connarus semidecandrus Jack. extract according to the present disclosure on the decreased expression of Bcl-2 and caspase-3 proteins or the increased expression of cleaved Caspase-3 proteins by testosterone treatment.

FIG. 9A shows a result of confirming an effect of a Connarus semidecandrus Jack. extract according to the present disclosure on the proliferation of human dermal papilla cells, FIG. 9B shows a result of confirming the proliferation of human dermal papilla cells after treatment with dihydrotestosterone (DHT) and treatment with a Connarus semidecandrus Jack. extract according to the present disclosure, and FIG. 9C shows a result of confirming the cytotoxicity of a Connarus semidecandrus Jack. extract according to the present disclosure on human dermal papilla cells.

FIG. 10 shows a result of confirming an effect of a Connarus semidecandrus Jack. extract according to the present disclosure on the activity of 5-α reductase.

FIG. 11A shows a result of confirming an effect of a Connarus semidecandrus Jack. extract according to the present disclosure on the expression of Bcl-2 mRNA decreased by DHT treatment, and FIG. 11B shows a result of confirming an effect of the Connarus semidecandrus Jack. extract according to the present disclosure on the expression of Caspase-9 mRNA increased by DHT treatment. In FIG. 11, ## indicates a value of p<0.01 compared to a normal group, and ** indicates a value of p<0.01 compared to a DHT treated group.

### BEST MODE

Hereinafter, the present disclosure will be described in detail.

The present disclosure provides a pharmaceutical composition for preventing or treating hair loss containing a Connarus semidecandrus Jack. extract.

Further, the present disclosure provides a pharmaceutical composition for promoting hair growth or hair regrowth containing a Connarus semidecandrus Jack. extract.

The Connarus semidecandrus Jack. is a plant found in Laos, Malaysia, Myanmar, Vietnam, India, Cambodia, and the Philippines, and is a medicinal plant known for anti-allergic, antioxidant, and antipyretic effects and used for treating bubonopanus.

However, much research is still needed on the efficacy of the Connarus semidecandrus Jack., and particularly, the effects of promoting hair growth or hair regrowth and preventing or treating hair loss have not been reported at present.

According to the present disclosure, it has been identified that when a testosterone-induced alopecia animal model is treated with the Connarus semidecandrus Jack. extract, hair growth was promoted, hair length and thickness were also increased, the expression of androgen receptors and apoptosis-related factors was inhibited, the survival and proliferation of human dermal papilla cells were promoted, and the activity of 5-α reductase was inhibited. Therefore, the Connarus semidecandrus Jack. extract according to the present disclosure can be provided in various forms of compositions for preventing or treating hair loss, and promoting hair growth or hair regrowth.

In the present disclosure, the extract may be extracted from at least one part selected from the group consisting of the whole plant, aerial part, roots, leaves, seeds, and flower of Connarus semidecandrus Jack.

In addition, the extract may be extracted with any one solvent selected from the group consisting of water, C1 to C4 alcohols, and a mixed solvent thereof, and preferably may be extracted with ethanol. More preferably, the extract may be extracted with 70% ethanol.

In one embodiment of the present disclosure, the Connarus semidecandrus Jack. extract was prepared by extracting the whole plant of Connarus semidecandrus jack. with 70% ethanol.

In addition, the Connarus semidecandrus Jack. extract may be extracted using a conventional extraction method in the art, such as a method using an extraction device such as room temperature extraction, supercritical extraction, high temperature extraction, high pressure extraction, or ultrasonic extraction, or a method using an adsorption resin including XAD and HP-20. In one embodiment of the present disclosure, the extract was extracted using room temperature extraction.

In addition, in order to facilitate processing, storage, and the like when preparing the Connarus semidecandrus Jack. extract of the present disclosure, a filtration process, a concentration and purification process, a drying process, a freezing process, etc. may be optionally added.

The filtration process may be performed by known filtration methods, and is not limited thereto, but may include, for example, filtration using a filter paper, a filter net, or a microfilter, centrifugation, and a separating funnel. The concentration process may be performed by known concentration methods, but is not limited thereto, and for example, the concentration may be performed by using rotary vacuum, sedimentation concentration, evaporation concentration, reduced pressure concentration, ultrafiltration, reverse osmosis, and centrifugation.

The drying process may be performed by known drying methods, but is not limited thereto, and for example, the drying process may be performed by freeze drying, spray drying, or hot air drying.

In the present disclosure, the “hair loss” may be included without special limitation if the number of hairs is less than the normal number beyond the level of normal hair growth. In the present disclosure, the hair loss may be at least one selected from the group consisting of male pattern alopecia, female pattern alopecia, alopecia areata, and telogen effluvium, but is not limited thereto. More specifically, the hair loss may include alopecia areata, hereditary androgenetic alopecia, telogen effluvium, anagen alopecia including traumatic, Trichoti lomania or pressure alopecia, pityriasis alopecia, alopecia syphlltiac, alopecia seborrhecia, symptomatic alopecia, non-scarring alopecia, scarring alopecia and congenital alopecia.

In the present disclosure, the “prevention” means all actions of inhibiting or delaying hair loss by administering the composition of the present disclosure, and the “treatment” means all actions of improving or beneficially changing symptoms of hair loss by the composition of the present disclosure.

In the present disclosure, the “hair growth” means hair sprouting, and the “hair regrowth” means hair growing. The effect of the composition according to the present disclosure also includes the effect of promoting hair growth or hair regrowth.

The pharmaceutical composition of the present disclosure may be formulated and used in the form of oral formulations such as powders, granules, tablets, capsules, suspensions, emulsions, syrups, aerosols, etc., external preparations, suppositories, and sterile injectable solutions according to conventional methods, respectively. The carrier, the excipient, and the diluent that may be included in the pharmaceutical composition may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil. When the composition is formulated, the formulation may be prepared by using diluents or excipients, such as a filler, an extender, a binder, a wetting agent, a disintegrating agent, and a surfactant, which are generally used. Solid formulations for oral administration include tablets, pills, powders, granules, capsules, and the like, and these solid formulations may be prepared by mixing at least one excipient, for example, starch, calcium carbonate, sucrose or lactose, gelatin, and the like with the Connarus semidecandrus Jack. extract of the present disclosure. Further, lubricants such as magnesium stearate and talc are used in addition to simple excipients. Liquid formulations for oral administration may correspond to suspensions, oral liquids, emulsions, syrups, and the like, and may include various excipients, for example, a wetting agent, a sweetener, an aromatic agent, a preservative, and the like, in addition to water and liquid paraffin which are commonly used as simple diluents. Formulations for parenteral administration include sterile aqueous solutions, non-aqueous solutions, suspensions, emulsions, lyophilized preparations, and suppositories. As the non-aqueous solution and the suspension, propylene glycol, polyethylene glycol, vegetable oil such as olive oil, injectable ester such as ethyl oleate, and the like may be used. As a base of the suppository, witepsol, macrogol, tween 61, cacao butter, laurinum, glycerogelatin, and the like may be used.

The dose of the pharmaceutical composition of the present disclosure will vary depending on the age, sex, and weight of a subject to be treated, a specific disease or pathological condition to be treated, the severity of the disease or pathological condition, a route of administration, and the judgment of a prescriber. The dose based on these factors is determined within a level of those skilled in the art, and in general, the dose is in the range of 0.01 mg/kg/day to about 2000 mg/kg/day. A more preferable dose is 0.1 mg/kg/day to 1000 mg/kg/day. The administration may be performed once a day or several times a day. The dose does not limit the scope of the present disclosure in any aspect.

The pharmaceutical composition of the present disclosure may be administered to mammals such as mice, livestock, and humans through various routes. The term “administration” used herein means providing a predetermined composition of the present disclosure to a subject by any suitable method. The pharmaceutical composition of the present disclosure may be administered through various routes including orally or parenterally, but is preferably administered parenterally, and more preferably, by topical application.

The pharmaceutical composition of the present disclosure may additionally include any compound or natural extract that has already been verified as safe and known to have effects of preventing or treating hair loss and promoting hair growth or hair regrowth, in order to prevent and alleviate hair loss, or increase and enhance the treatment effect of hair loss, in addition to the Connarus semidecandrus Jack. extract according to the present disclosure.

Further, the pharmaceutical composition of the present disclosure may be used alone or in combination with methods of using surgery, radiation therapy, hormone therapy, chemotherapy, and biological response modifiers for treatment of hair loss.

The hair to which the composition of the present disclosure is applied includes hair roots and hair follicles of the head, hair, eyelashes and eyebrows, beard, armpits, pubic hair, and all parts of the body with hair roots and hair follicles.

Further, the present disclosure provides a quasi-drug composition for preventing or alleviating hair loss containing a Connarus semidecandrus Jack. extract.

Further, the present disclosure provides a quasi-drug composition for promoting hair growth or hair regrowth containing a Connarus semidecandrus Jack. extract.

In the present disclosure, the “quasi-drug” means fibers, rubber products or similar products thereto used for the purpose of treating, alleviating, handling or preventing a disease of a human or animal, products having a weak effect on the human body or not directly acting on the human body, excluding instruments or machines, and similar products thereto, and articles corresponding to one of preparations to be used for sterilization, insecticide, and similar uses thereto for the purpose of preventing infection. The “quasi-drug” means articles used for the purpose of diagnosing, treating, alleviating, handling or preventing diseases of humans or animals, excluding instruments, machines or devices, and articles used for the purpose of exerting a pharmacological effect on the structure and function of humans or animals, excluding instruments, machines or devices, and also includes external skin preparations and personal care products.

When the composition of the present disclosure is included in quasi-drugs for the purpose of preventing or alleviating hair loss and promoting hair growth or hair regrowth, the composition may be included and used as it is or together with other quasi-drug ingredients, and may be used appropriately according to a conventional method. The mixing amount of the active ingredients may be appropriately determined depending on the purpose of use.

The quasi-drugs of the present disclosure are not particularly limited thereto, but may be manufactured and used in the form of, for example, creams, lotions, aerosols, shampoos, gels or packs.

The creams, ointments, shampoos, gels or packs include bases such as white petrolatum, yellow petrolatum, lanolin, bleached beeswax, cetanol, stearyl alcohol, stearic acid, hydrogenated oils, gelled hydrocarbons, polyethylene glycol, liquid paraffin, squalane, etc.; solvents and solubilizers such as oleic acid, isopropyl myristate, glycerin triisooctanoate, crotamiton, diethyl sebacate, diisopropyl adipate, hexyl laurate, fatty acids, fatty acid esters, aliphatic alcohols, vegetable oils, etc.; antioxidants such as tocopherol derivatives, L-ascorbic acid, dibutylhydroxytoluene, butylhydroxyanisole, etc.; preservatives such as parahydroxybenzoic acid ester, etc.; moisturizers such as glycerin, propylene glycol, sodium hyaluronate, etc.; surfactants such as polyoxyethylene derivatives, glycerin fatty acid esters, sucrose fatty acid esters, sorbitan fatty acid esters, propylene glycol fatty acid esters, lecithin, etc.; and thickeners such as carboxyvinyl polymers, xanthan gum, carboxymethyl cellulose, carboxymethyl cellulose sodium salts, hydroxypropyl cellulose, hydroxypropyl methyl cellulose, etc.

The aerosols may be mixed with bases such as white petrolatum, yellow petrolatum, lanolin, bleached beeswax, cetanol, stearyl alcohol, stearic acid, hydrogenated oils, gelled hydrocarbons, polyethylene glycol, liquid paraffin, squalane, etc.; solvents and solubilizers such as oleic acid, isopropyl myristate, diisopropyl adipate, isopropyl sebacate, glycerin triisooctanoate, crotamiton, diethyl sebacate, hexyl laurate, fatty acids, fatty acid esters, aliphatic alcohols, vegetable oils, etc.; antioxidants such as tocopherol derivatives, L-ascorbic acid, dibutylhydroxytoluene, butylhydroxyanisole, etc.; preservatives such as parahydroxybenzoic acid ester, etc.; moisturizers such as glycerin, propylene glycol, sodium hyaluronate, etc.; surfactants such as polyoxyethylene derivatives, glycerin fatty acid esters, sucrose fatty acid esters, sorbitan fatty acid esters, propylene glycol fatty acid esters, lecithin, etc.; and thickeners such as carboxyvinyl polymers, xanthan gum, carboxymethyl cellulose, carboxymethyl cellulose sodium salts, hydroxypropyl cellulose, hydroxypropyl methyl cellulose, etc., and further include various stabilizers, buffers, coupling agents, suspending agents, emulsifiers, fragrances, preservatives, solubilizers, and other suitable additives, which are used in the preparation of ointments, creams, gels, suspensions, emulsions, solutions, and lotions. In addition, stabilizers, preservatives, absorption promoters, pH adjusters, and other suitable additives may be mixed as needed.

Further, the present disclosure provides a cosmetic composition for preventing or alleviating hair loss containing a Connarus semidecandrus Jack. extract.

Further, the present disclosure provides a cosmetic composition for promoting hair growth or hair regrowth containing a Connarus semidecandrus Jack. extract.

The ingredients included in the cosmetic composition of the present disclosure may include ingredients commonly used in the cosmetic composition in addition to the Connarus semidecandrus Jack. extract, and include, for example, conventional auxiliary agents and carriers, such as stabilizers, solubilizers, vitamins, pigments, and fragrances.

The cosmetic composition of the present disclosure may be prepared in any formulation commonly prepared in the art, and examples thereof may include emulsions, creams, toners, packs, foundations, lotions, essences, hair cosmetics, etc.

Specifically, the cosmetic composition of the present disclosure includes formulations of skin lotion, skin softener, skin toner, astringent, lotion, milk lotion, moisture lotion, nourishing lotion, massage cream, nourishing cream, moisture cream, hand cream, foundation, essence, nourishing essence, pack, soap, cleansing foam, cleansing lotion, cleansing cream, body lotion, and body cleanser.

More preferably, the cosmetic composition of the present disclosure may be formulations of hair tonic, hair cream, hair lotion, hair shampoo, hair rinse, hair conditioner, hair spray, hair aerosol, pomade, powder gel, hair pack, hair treatment, eyebrow tonic, eyelash tonic or eyelash nutrient, but is not limited thereto.

When the formulation of the present disclosure is the paste, cream, or gel, as a carrier ingredient, animal oils, vegetable oils, wax, paraffin, starch, tragacanth, cellulose derivatives, polyethylene glycol, silicone, bentonite, silica, talc, zinc oxide or the like may be used.

When the formulation of the present disclosure is the powder or spray, as the carrier ingredient, lactose, talc, silica, aluminum hydroxide, calcium silicate or polyamide powder may be used, and particularly, in the case of the spray, a propellant such as chlorofluorohydrocarbon, propane/butane or dimethyl ether may be further included.

When the formulation of the present disclosure is the solution or emulsion, as the carrier ingredient, a solvent, a solubilizer or an emulsifier may be used. For example, the carrier ingredient includes water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or fatty acid ester of sorbitan.

When the formulation of the present disclosure is the suspension, as the carrier ingredient, a liquid diluent such as water, ethanol or propylene glycol, a suspension such as ethoxylated isostearyl alcohol, polyoxyethylene sorbitol ester and polyoxyethylene sorbitan ester, microcrystalline cellulose, aluminum metahydroxide, bentonite, agar, tragacanth, or the like may be used.

When the formulation of the present disclosure is the surfactant-containing cleansing, as the carrier ingredient, aliphatic alcohol sulfate, aliphatic alcohol ether sulfate, sulfosuccinic acid monoester, isethionate, imidazolinium derivatives, methyltaurate, sarcosinate, fatty acid amide ether sulfate, alkylamido betaine, aliphatic alcohol, fatty acid glyceride, fatty acid diethanolamide, vegetable oil, lanolin derivatives, ethoxylated glycerol fatty acid ester, or the like may be used.

In addition to the active ingredients, the cosmetic composition of the present disclosure may further contain one or more ingredients that help in alleviating skin condition, including ingredients for preventing, alleviating or treating hair loss, and promoting hair growth or hair regrowth, which have the same or similar function. The ingredients include hyaluronic acid, butylene glycol, glycerin, amino acid, trehalose, kojic acid and derivatives thereof, arbutin, ascorbic acid and derivatives thereof, hydroquinone and derivatives thereof, resorcinol, 2,7-dinitroindazole, adenosine, retinol, retinyl palmitate, polyethoxylated retinamide, yeast, dipeptide, palmitoyl oligopeptide & palmitoyl tripeptide-7, acetyl hexapeptide, epidermal growth factor (EGF), or plant extracts such as tangerine, rice, licorice, shea butter, aloe vera, coconut, olive, and avocado, but are not limited thereto.

In the present disclosure, when the Connarus semidecandrus Jack. extract is used as a cosmetic composition for preventing or alleviating hair loss or a cosmetic composition for promoting hair growth or hair regrowth, the Connarus semidecandrus Jack. extract may be added in an amount of 0.001 to 50.0 wt %, and preferably 0.005 to 10.0 wt % based on the total weight of the cosmetic composition. When the content is less than 0.001 wt %, it is difficult to expect a practical hair growth promoting effect, and when the content exceeds 50 wt %, there may be a problem that the manufacturing cost increases compared to a cosmetic effect.

In addition, the composition of the present disclosure for preventing or alleviating hair loss and promoting hair growth or hair regrowth may be used for pets depending on a formulation. For example, the composition of the present disclosure may be prepared in various forms such as solutions, sol-gels, emulsions, oil, wax, aerosols, etc., such as pet shampoos and pet rinses, and prepared by adding a neutral detergent that is less irritating to the skin of the pets and has excellent moisturizing power.

Further, the present disclosure provides a food composition for preventing or alleviating hair loss containing a Connarus semidecandrus Jack. extract.

Further, the present disclosure provides a food composition for promoting hair growth or hair regrowth containing a Connarus semidecandrus Jack. extract.

Further, the present disclosure provides a health functional food composition for preventing or alleviating hair loss containing a Connarus semidecandrus Jack. extract.

Further, the present disclosure provides a health functional food composition for promoting hair growth or hair regrowth containing a Connarus semidecandrus Jack. extract.

The food composition according to the present disclosure includes all forms, such as functional food, nutritional supplements, health food, health supplements, and food additives. The type of food composition may be formulated in any one form selected from the group consisting of powders, tablets, capsules, pills, and liquids according to a conventional method known in the art, but is not limited thereto. The food composition may be prepared in various forms using methods known in the art.

For example, as the health food, the Connarus semidecandrus Jack. extract of the present disclosure may be granulated, encapsulated, and powdered and then ingested, or prepared and then drunken in the form of tea, juice, and drinks. In addition, the Connarus semidecandrus Jack. extract of the present disclosure may be mixed with known substances or active ingredients known to have the activity for preventing, alleviating or treating hair loss, and promoting hair growth or hair regrowth to be prepared in the form of composition.

In addition, the functional food may be prepared by adding the Connarus semidecandrus Jack. extract of the present disclosure to beverages (including alcoholic beverages), fruits and processed foods thereof (e.g., canned fruit, bottled food, jam, marmalade, etc.), fish, meat and processed foods thereof (e.g., ham, sausage, corned beef, etc.), bread and noodles (e.g., udon, buckwheat noodles, ramen, spaghetti, macaroni, etc.), fruit juice, various drinks, cookies, sweets, dairy products (e.g., butter, cheese, etc.), edible vegetable oil, margarine, vegetable protein, retort food, frozen food, various seasonings (e.g., soybean paste, soy sauce, sauce, etc.), etc.

In addition, the food composition of the present disclosure may include conventional food additives, and the suitability as the “food additive” is determined by the specifications and standards for the corresponding item in accordance with the general rules of the Food Additive Codex, general test methods, and the like approved by the Food and Drug Administration, unless otherwise specified. The items disclosed in the “Food Additives Codex” may include, for example, chemical composites such as ketones, glycine, calcium citrate, nicotinic acid, cinnamic acid, etc., natural additives such as desensitizing dye, licorice extract, crystal cellulose, Kaoliang color, guar gum, etc., and mixed formulations such as sodium L-glutamic acid formulations, alkali agents for addition to noodles, preservative formulations, tar color formulations, etc.

In the food composition of the present disclosure, the Connarus semidecandrus jack. extract may preferably be included in an amount of 0.00001 to 50 wt % based on the food composition. If the content is less than 0.00001 wt %, the effect thereof is insignificant, and if the content exceeds 50 wt %, an increase in effect compared to the amount used is minimal, which is uneconomical.

In addition, in order to use the Connarus semidecandrus jack. extract of the present disclosure in the form of food additives, the Connarus semidecandrus jack. extract may be prepared and used in the form of tablets, capsules, powder, granules, liquid, pills, etc.

When the composition of the present disclosure is prepared as beverages, like general beverages, the composition may include various flavoring agents or natural carbohydrates as an additional ingredient. The above-mentioned natural carbohydrates may be used with monosaccharides such as glucose and fructose, disaccharides such as maltose and sucrose, natural sweeteners such as dextrin and cyclodextrin, synthetic sweeteners such as saccharin and aspartame, and the like. A ratio of the natural carbohydrates may be generally about 0.01 to 10 g, preferably about 0.01 to 0.1 g per 100 ml of the composition of the present disclosure.

In addition, the composition of the present disclosure may include various nutrients, vitamins, electrolytes, flavoring agents, coloring agents, pectic acid and salts thereof, alginic acid and salts thereof, organic acid, a protective colloidal thickener, a pH adjusting agent, a stabilizer, a preservative, glycerin, alcohols, a carbonic acid agent used in a carbonated drink, or the like. In addition, the composition of the present disclosure may include pulps for preparing natural fruit juices, fruit juice beverages or vegetable beverages. These ingredients may be used independently or in combination. Although the ratio of these additives is not greatly important, generally, the ratio thereof is selected in the range of 0.01 to 0.1 parts by weight per 100 parts by weight of the composition of the present disclosure.

As used herein, the “health supplement” or “health functional food” refers to food prepared and processed using raw materials or ingredients with functionality, which are useful for the human body according to the Health Functional Foods Act. The “functionality” means ingestion for the purpose of adjusting nutrients for the structures and functions of the human body or obtaining useful effects on health applications such as physiological actions.

Further, the present disclosure provides a method for treating hair loss including treating a subject with a Connarus semidecandrus Jack. extract.

Further, the present disclosure provides a method for promoting hair growth or hair regrowth including treating a subject with a Connarus semidecandrus Jack. extract.

The subject is preferably a mammal, including a human, and may be applied without limitation to any subject requiring treatment of hair loss, hair growth, or hair regrowth.

Further, the Connarus semidecandrus Jack. extract according to the present disclosure may be treated in combination with drugs or treatment methods for treatment of hair loss, hair growth or hair regrowth. When the Connarus semidecandrus Jack. extract of the present disclosure is treated in combination, the Connarus semidecandrus Jack. extract may be treated simultaneously or sequentially with other drugs or treatment methods for treatment of hair loss, hair growth or hair regrowth.

The above-described contents of the present disclosure are equally applied to each other unless otherwise contradict each other, and those appropriately modified and implemented by those skilled in the art are also included in the scope of the present disclosure.

### Modes

Hereinafter, the present disclosure will be described in detail through Examples, but the scope of the present disclosure is not limited only to the Examples below.

### Experimental Example 1. Materials and Reagents

Human hair follicle dermal papilla cells (HDPs) were purchased from the American Type Culture Collection (ATCC) (Rockville, MD, USA). Testosterone, finasteride, sesame oil, and dimethyl sulfoxide (DMSO) were purchased from Sigma (St. Louis, MO, USA). A RPMI 1640 medium, trypsin (0.25%), and antibiotics (penicillin-streptomycin) solution were purchased from HyClone Laboratories (Logan, UT, USA). A CEFOgro™ Human Dermal Papilla Growth Medium as a HDP growth medium was obtained from CEFO Co. (Seoul, Korea). Fetal Bovine Serum (FBS) was purchased from Gibco (Grand Island, NY, USA). Phosphate-buffered saline (PBS) was purchased from Samchun Pure Chemical Co. (Gyeonggi-do, Korea). A TRIzol reagent was purchased from Molecular Research Center, Inc. (Cincinnati, OH, USA). Antibodies for Bcl-2 (catalog number #2876), Bax (catalog number #2772), Caspase-3 (catalog number #9662), and Cleaved Caspase 3 (catalog number #9661) were obtained from Cell Signaling Technology (Beverly, MA, USA). In addition, antibodies for glyceraldehyde 3-phosphate dehydrogenase (GAPDH) (catalog number #sc-47724) and an androgen receptor (AR) (catalog number #sc-7305) were obtained from Santa Cruz Biotechnology, Inc. (Dallas, TX, USA).

### Experimental Example 2. Experimental Animals and Cell Culture

C57BL/6 mice (male, five weeks old) were purchased from Orient Bio. In addition, the mice were raised with sufficient water and food. The animal experiment of the present disclosure was performed in accordance with the guidelines (SKKUIACUC-2021 Jul. 18-1) of the Institutional Animal Care and Use Committee (IACUC) at Sungkyunkwan University. The HDP cells were cultured in a HDP growth medium under conditions of 37° C. and 5% CO₂.

### Experimental Example 3. Fabrication of Animal Model

A testosterone-induced androgenetic alopecia model was fabricated using C57BL/6 mice. C57BL/6 mice (n=6) were divided into the following five groups: Normal group, Vehicle control group: Experimental group treated with 40 μg/kg/week testosterone, Experimental group treated with 5 mg/day Cs-EE (Connarus semidecandrus Jack. extract according to the present disclosure), Experimental group treated with 10 mg/day Cs-EE, and Positive control group: Experimental group treated with 5 mg/kg/day finasteride. To confirm a hair growth promoting effect of Cs-EE, the dorsal part of each mouse was treated with hair removal cream and shaved using an electric clipper twice; seven and three days before the first testosterone treatment.

40 μg/kg of testosterone was dissolved in sesame oil and then subcutaneously injected once a week four times. Thereafter, in the case of the Cs-EE-treated experimental group, Cs-EE diluted with PBS and DMSO (ratio of 1:1) was applied to the back part of the mouse once every day for three weeks. As the positive control group, 5 mg of finasteride was treated instead of Cs-EE of the present disclosure. Two days after the testosterone injection, mice in all experimental groups were euthanized. Thereafter, the dorsal hairs were isolated from each experimental group to confirm hair type frequency and hair thickness.

### Experimental Example 4. Hair Cover Skin Ratio, Hair Type Frequency, and Hair Thickness Analysis

After shaving the hairs in the androgenetic alopecia animal model of Example 3 above, pictures of the dorsal skin were taken every week. In addition, the scores of hair growth areas on the dorsal skin were selected from 1 to 10 depending on their colors using ImageJ to evaluate the growth degree of hair. Then, up to 350 hairs per mouse were collected to identify four types (guard hair, awl hair, auchene hair and zigzag hair) of mouse hair.

### Experimental Example 5. Hematoxylin & Eosin Staining

The skin of the androgenetic alopecia animal model of Example 3 above was fixed in 3.7% formalin for two days at 4° C. and then embedded in paraffin. After sectioning at 4 μm, the hair loss was confirmed by staining using hematoxylin and eosin.

### Experimental Example 6. Ex Vivo Culture of Hair Follicles in Androgenetic Alopecia Animal Model

The androgenetic alopecia animal model of Example 3 above was euthanized to obtain vibrissae hair follicles (n=15). The obtained hair follicles were cultured in a HDP growth medium under the conditions of 37° C. and 5% CO₂. One day after media suction, either DMSO or Cs-EE 100 μg/mL was treated to the hair follicles, and the hair length was measured at 0, 8, 16, 24, and 48 hours after seeding, respectively.

### Experimental Example 7. Quantitative Real-Time PCR

RNA was extracted from the skin tissue of the androgenetic alopecia animal model of Example 3 above and total RNA was extracted from HDP cells treated with 100 nM of DHT for 24 hours after pre-treatment with 25, 50, and 100 μg/mL of Cs-EE using a TRIzol reagent. Using this, cDNA was synthesized using a cDNA synthesis kit (Thermo Fisher Scientific) according to the manufacturer's instructions. The mRNA levels of Bcl-2, Bax, and Caspase-9 were determined by real-time PCR using SYBR premix Ex Taq (using primers in Tables 1 and 2). At this time, the expression level of each gene was calculated based on GAPDH expression.

TABLE 1

| PCR | | | | SEQ ID |
|---|---|---|---|---|
| type | Gene name | Direction | Sequence (5′-3′) | NO: |
| Real- | Bcl-2 | Forward | ATCGCCCTGTGGATGACTGAGT | 1 |
| time | (Human) | Reverse | GCCAGGAGAAATCAAACAGAGGC | 2 |
| PCR | Caspase 9 | Forward | AGGCAAGCAGCAAAGTTGTC | 3 |
| | (Human) | Reverse | GTCTTTCTGCTCGACATCACCA | 4 |
| | GAPDH | Forward | GACAGTCAGCCGCATCTTCT | 5 |
| | (Human) | Reverse | GCGCCAATACGACCAAATC | 6 |

TABLE 2

| | | | | SEQ ID |
|---|---|---|---|---|
| PCR type | Gene name | Direction | Sequence (5′-3′) | NO: |
| Real-time | Bcl-2 | Forward | GAGTACCTGAACCGGCATCT | 7 |
| PCR | (Mouse) | Reverse | GAAATCAAACAGAGGTCGCA | 8 |
| | Bax | Forward | GAACCATCATGGGCTGGACA | 9 |
| | (Mouse) | Reverse | GGAGAGGAGGCCTTCCCAG | 10 |
| | Caspase 9 | Forward | CGAGAACTACCGCAGGAAGC | 11 |
| | (Mouse) | Reverse | CTGTCGTATTCCCGCGATCC | 12 |
| | GAPDH | Forward | TGTGAACGGATTTGGCCGTA | 13 |
| | (Mouse) | Reverse | ACTGTGCCGTTGAATTTGCC | 14 |

### Experimental Example 8. Western Blotting

The skin tissues of the androgenetic alopecia animal model of Example 3 above were ground with liquid nitrogen and then stored at −70° C. Thereafter, the tissues were lysed using a lysis buffer (including 50 mM Tris-HCl (pH 7.4), 120 mM NaCl, 25 mM β-glycerol phosphate (pH 7.5), 20 mM NaF (sodium fluoride), 2% NP-40 (Nonidet P-40), and protease inhibitors). After subsequent sonication, the lysates were centrifuged at 4° C. and 12,000×g for 3 minutes. The supernatants were obtained for Western blotting analysis and analyzed by 10-15% SDS-polyacrylamide gel electrophoresis.

The levels of AR, Bcl-2, Caspase-3, cleaved Caspase-3, and β-actin were confirmed using the corresponding antibodies by a previously known Western blotting method, respectively.

### Experimental Example 9. 5-α Reductase Activity analysis

The whole liver of a 10-week-old male Sprague-Dawley rat was extracted and lysed with a lysis buffer (including 7.5 nM K₂HPO₄, 3.25 nM KH₂PO₄, 1 mM dithiothreitol (DTT), 32 mM sucrose, 0.2 mM phenylmethylsulfonyl fluoride (PMSF), and additional protease inhibitors) to obtain 5-α reductase.

The 5-α reductase was immediately added with 34 mM of nicotinamide adenine dinucleotide phosphate (NADPH), 0.4 mM of testosterone, and Mcilvaine buffer (pH 5.0) to 4 μL of an enzyme extract that had been treated with serial concentrations of Cs-EE. Thereafter, the reaction was stopped by heating at 80° C. for 5 minutes. Further, absorbance was measured at 340 nm to detect oxidation of NADPH.

### Experimental Example 10. Cell Viability Analysis

HDP cells were cultured at a concentration of 5×10⁴ cells/mL in a 96 well plate.

50 μl of Cs-EE was dose-dependently treated into each well. After 24 hours, 10 μl of 3-(4,5-dimethylthiazol-2-yl)-2,5-diphenyl tetrazolium bromide (MTT) was added to each well and reacted for 3 hours. The reaction was stopped by adding 100 μl of 10% SDS in 0.01 M HCl to each well and formazan was dissolved for 24 hours. Then, the absorbance of MTT formazan was measured at 540 nm.

### Experimental Example 11. Cell Proliferation Analysis

Cell proliferation analysis was performed in the presence or absence of dihydrotestosterone (DHC). HDP cells were cultured at a concentration of 3× 10³ cells/mL in a 96-well plate, and cell viability was confirmed after 0, 24, 48, and 72 hours of Cs-EE treatment. The optical density was detected at 540 nm.

### Experimental Example 12. Statistical Analysis

All experiments were at least twice repeated independently for statistical comparisons of the mean±standard deviation (SD) value. For statistical comparison, the data were analyzed with a t-test, and the P-values of less than 0.05 were considered significant.

### Example 1. Preparation of Connarus semidecandrus Jack. Extract

A Connarus semidecandrus Jack. extract was obtained from the National Institute of Biological Resources under the Ministry of Environment and prepared by extraction with 70% ethanol. More specifically, the Connarus semidecandrus Jack. extract was prepared as follows. First, the whole plant was treated with 70% ethanol at a temperature of 20 to 22° C. for 24 hours and then extracted. Thereafter, the ethanol was removed and concentrated under vacuum conditions of 10 hPa and 40° C. using a rotary evaporator (Buchi Labortechnik AG, Purfil, Switzerland). Thereafter, the solvent was further completely removed under the conditions of 5 mTorr and −85° C., and the extract (named Cs-EE) was freeze-dried and stored.

### Example 2. Confirmation of Hair Growth Promoting and Hair Thickening Effects of Cs-EE in Alopecia Animal Model

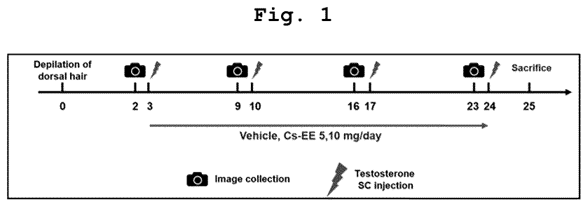

An experimental timeline for confirming hair growth promoting and hair thickening effects of Cs-EE according to the present disclosure in an androgenetic alopecia animal model induced by testosterone was as shown in FIG. 1.

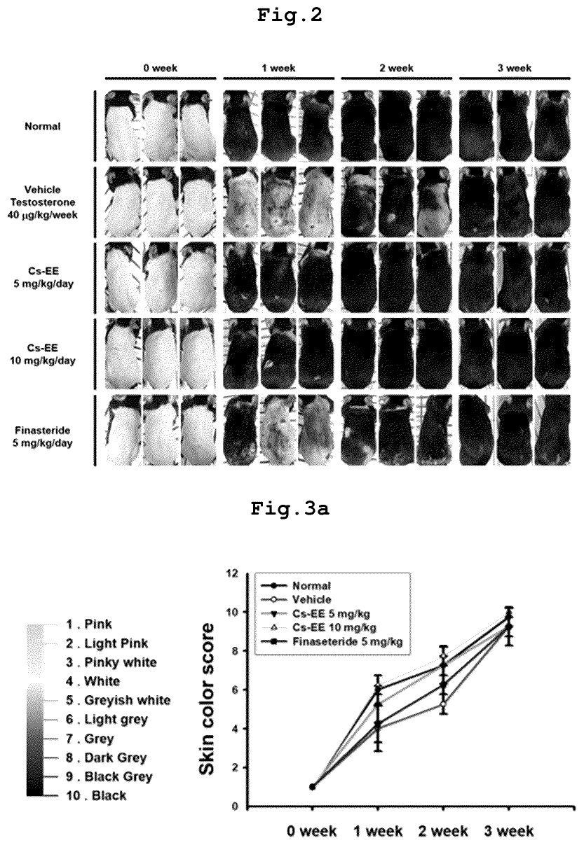

As shown in FIG. 2, an experimental group treated with Cs-EE 3 weeks after shaving showed significant regrowth with skin color almost completely restored. In addition, the experimental group was restored faster than a vehicle experimental group (Vehicle Testosterone 40 μg/kg/week experimental group) as a negative control group and a finasteride-treated experimental group (Finasteride 5 mg/kg/day experimental group) as a positive control group. On 1 week, most of the shaved skin in the negative control group (Vehicle) and the finasteride experimental group remained hairless, whereas the experimental group treated with Cs-EE according to the present disclosure showed clear hair growth.

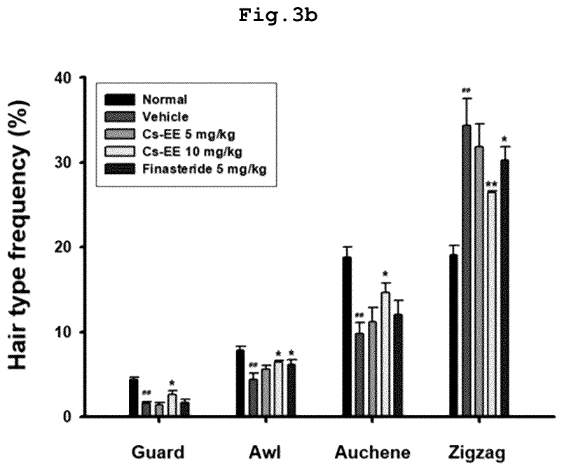

Based on the collected photographs in FIG. 2, skin color scores were measured by determining a color from pink to black, which was shown in FIG. 3A. As a result, the skin color score of the Cs-EE treated experimental group showed a similar trend to that of the normal group (Normal). In addition, the scores were significantly higher at all time points than the negative control group (Vehicle) or the finasteride treated experimental group. Through this, it was confirmed that the Cs-EE of the present disclosure had a stable hair regrowth promoting effect similar to that of the normal group.

In addition, as shown in FIG. 3B, it was confirmed that the hair type frequency also changed significantly. In the negative control group (Vehicle), it was confirmed that the number of thin and zigzag-shaped hairs increased due to testosterone treatment. However, it was confirmed that the hair loss was alleviated by Cs-EE treatment according to the present disclosure, and in particular, the effect of the experimental group treated with 10 mg/kg of Cs-EE was the best.

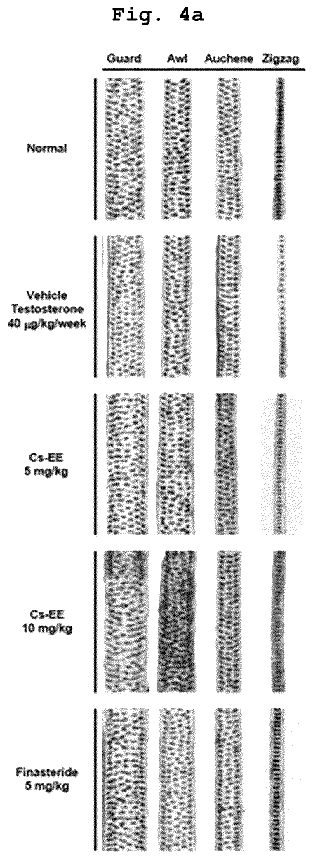

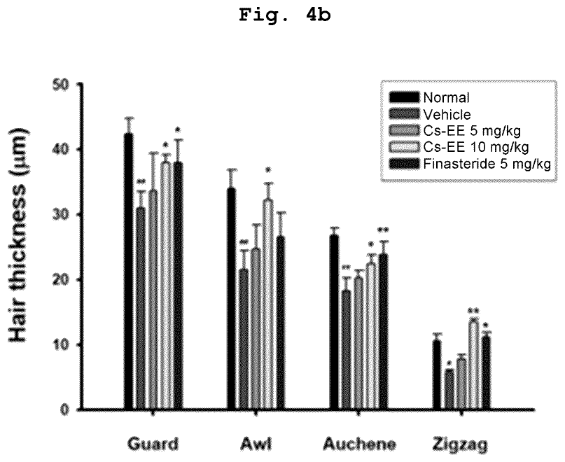

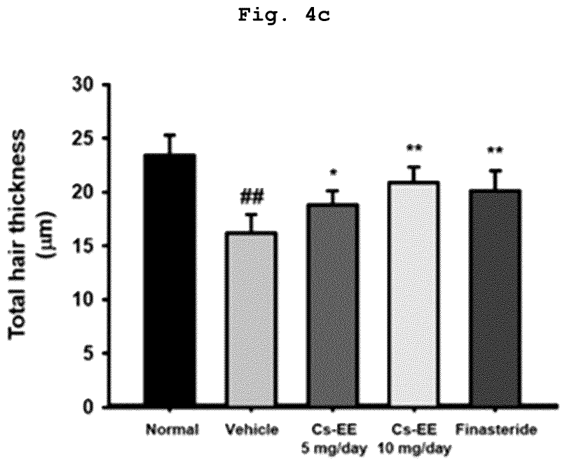

In addition, photographs of the hair type of each experimental group were shown in FIG. 4A. As a result, it was confirmed that the hair thickness of the negative control group (Vehicle) was thinner than that of the normal group, whereas the hair thickness of the Cs-EE treated experimental group according to the present disclosure was increased.

In addition, the thickness of each hair type was measured based on the hair type photograph of each experimental group and was shown in FIG. 4B. As a result, it was confirmed that the Cs-EE treated experimental group according to the present disclosure restored the hair thickness that had been reduced by testosterone. In addition, as shown in FIG. 4C, it was confirmed that the total hair thickness of the Cs-EE treated experimental group according to the present disclosure was also restored.

### Example 3. Histological Analysis of Mouse Hair Follicles and Confirmation of Hair Protection and Growth Promoting Effects of Cs-EE in In Vitro Culture

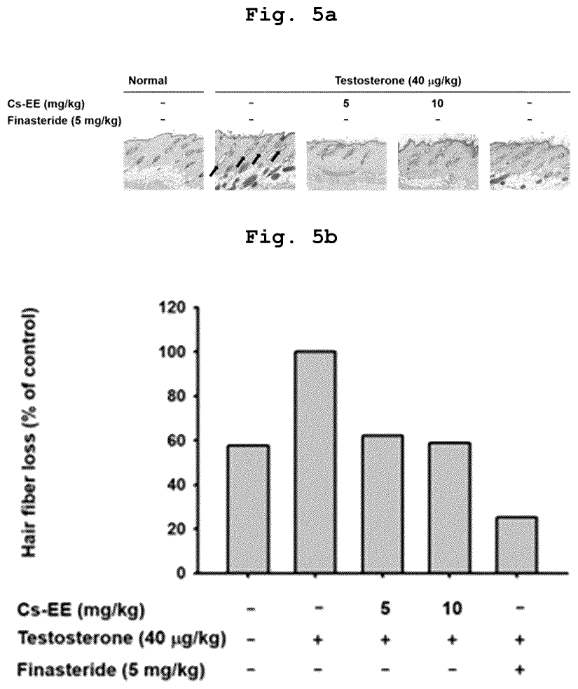

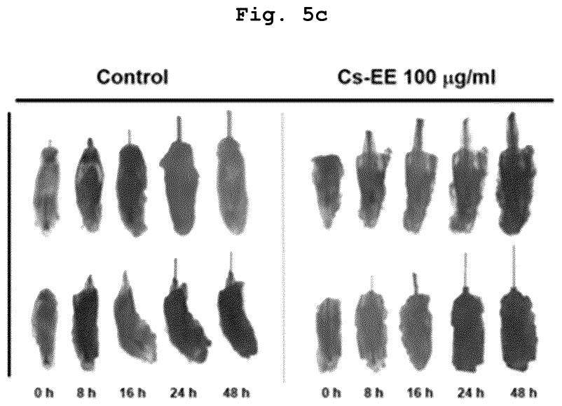

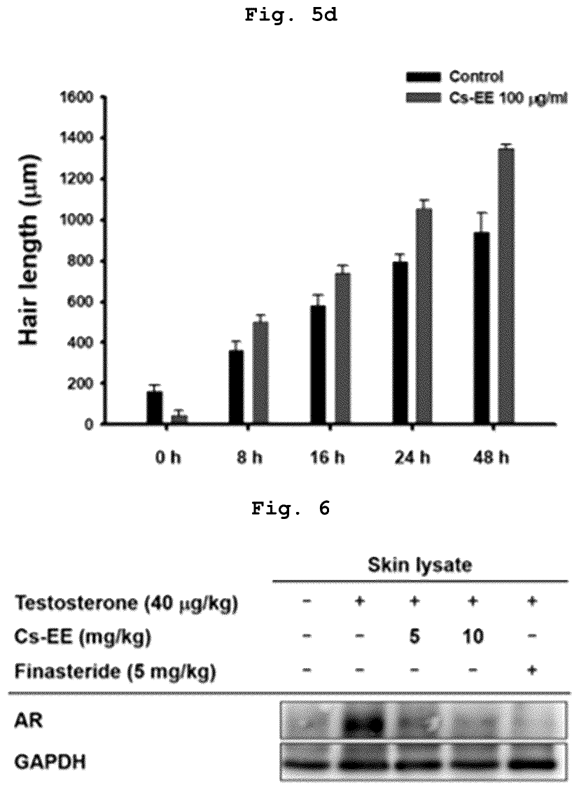

The hair growth promoting effect of Cs-EE according to the present disclosure was confirmed through histological analysis of mouse hair follicles. As shown in FIG. 5A, the hair follicles of the testosterone-treated experimental group showed empty spaces without hair fibers (indicated by black arrows). However, as shown in FIGS. 5A and 5B, it was confirmed that the Cs-EE treated experimental group promoted the development of new hair fibers on the epidermal surface.

In addition, the hair growth promoting effect of Cs-EE according to the present disclosure was confirmed even in in vitro culture of hair follicles. As shown in FIGS. 5C and 5D, as a result of culturing the hair follicles for 48 hours with or without treatment with 100 μg/ml of Cs-EE, when not treated with Cs-EE, the hair length reached less than 1000 μm, but when treated with Cs-EE, the hair length increased to 1200 μm or more.

Therefore, it was confirmed that Cs-EE according to the present disclosure inhibited testosterone-induced hair loss and induced hair growth.

### Example 4. Confirmation of Mechanism of Cs-EE on Hair Protection and Growth Promoting Effects

To confirm the mechanism of the hair loss prevention effect of Cs-EE, the level of AR protein in the skin of an androgenetic alopecia animal model induced by testosterone and the like was confirmed through Western blot. As a result, as shown in FIG. 6, it was confirmed that Cs-EE according to the present disclosure inhibited the AR expression to have an anti-androgenic effect.

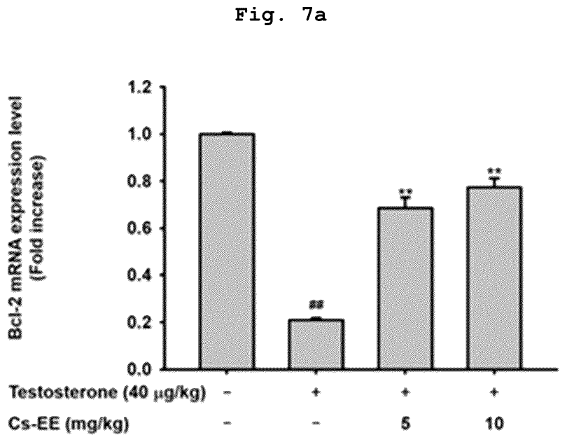

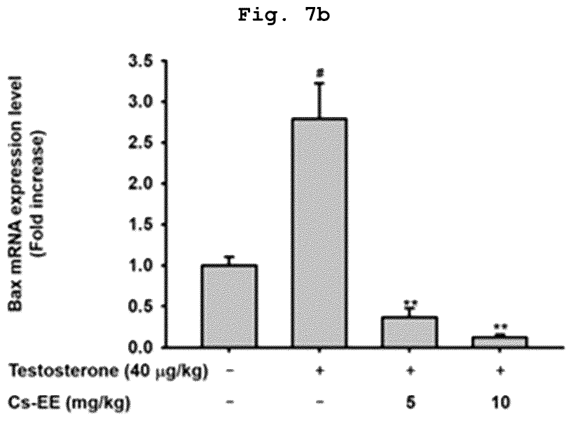

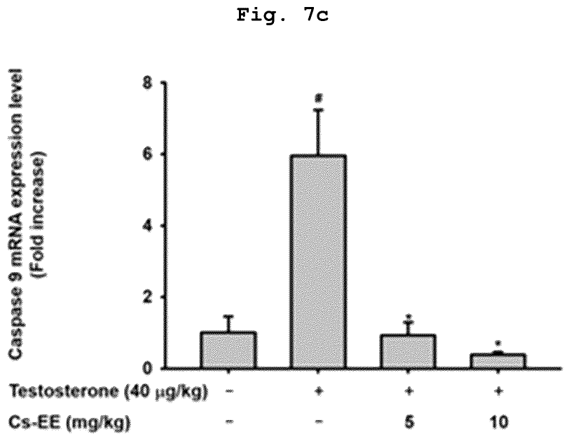

In hair loss induction, premature termination of hair follicle growth due to apoptosis is known to be one of important factors. Here, the mRNA expression of Bcl-2, Bax, and Caspase-9 was confirmed in skin tissues, and a result was shown in FIG. 7. Cs-EE according to the present disclosure increased the mRNA expression level of Bcl-2 (see FIG. 7A), but significantly decreased the mRNA expression of Bax and Caspase-9, which were genes with pro-apoptotic properties (see FIGS. 7B and 7C).

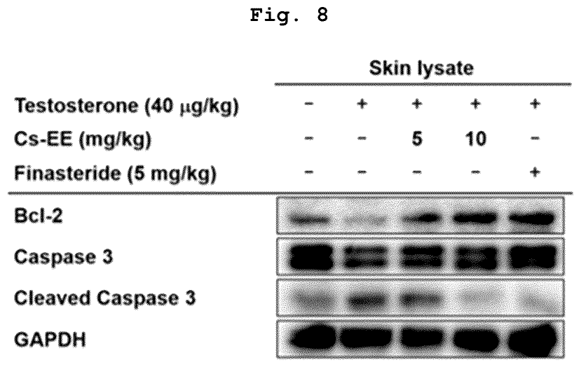

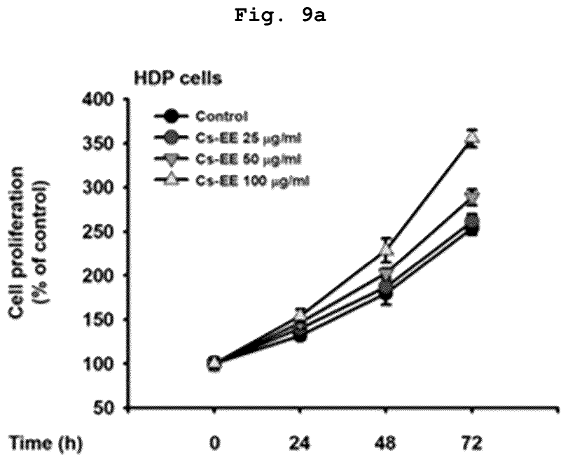

In addition, as shown in FIG. 8, it was confirmed that the expression of cleaved caspase-3 increased by testosterone was reduced by Cs-EE treatment according to the present disclosure. In addition, it was confirmed that the expression of Bcl-2, which had been reduced by testosterone, was restored by Cs-EE treatment according to the present disclosure.

Therefore, it was confirmed that the hair protection and growth promoting effects of Cs-EE according to the present disclosure were derived from the down-regulation of the AR and apoptosis pathways.

### Example 5. Confirmation of Effect of Cs-EE on Proliferation and Survival of HDP Cells

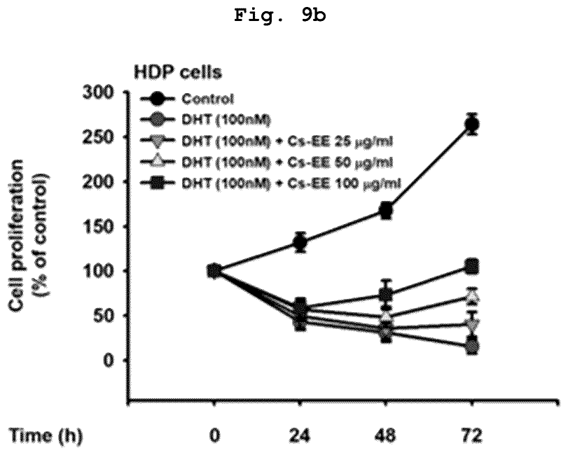

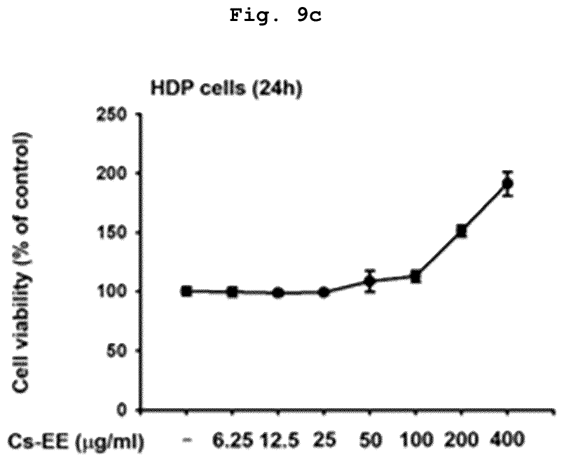

The effect of Cs-EE according to the present disclosure on the proliferation of HDP cells was confirmed. As a result, as shown in FIG. 9A, it was confirmed that HDP proliferation increased in the presence of Cs-EE according to the present disclosure. In addition, as shown in FIG. 9B, 100 nM DHT was cytotoxic to HDP cells, but the HDP cell proliferation was restored up to 100% by co-treatment with Cs-EE according to the present disclosure.

Through this, it was confirmed that Cs-EE according to the present disclosure may block an androgen pathway and prevent the death of HDP cells.

In addition, the cytotoxicity of Cs-EE according to the present disclosure to the HDP cells was confirmed. As a result, as shown in FIG. 9C, Cs-EE according to the present disclosure not only did not affect cell viability even at a concentration of 100 μg/ml, but also significantly increased the cell viability when treated at a concentration of 100 μg/ml or higher.

### Example 6. Confirmation of Effect of Cs-EE on 5-α Reductase and Anti-Apoptotic Process in HDP Cells

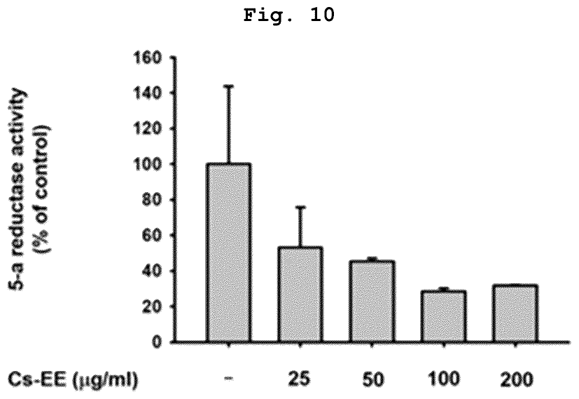

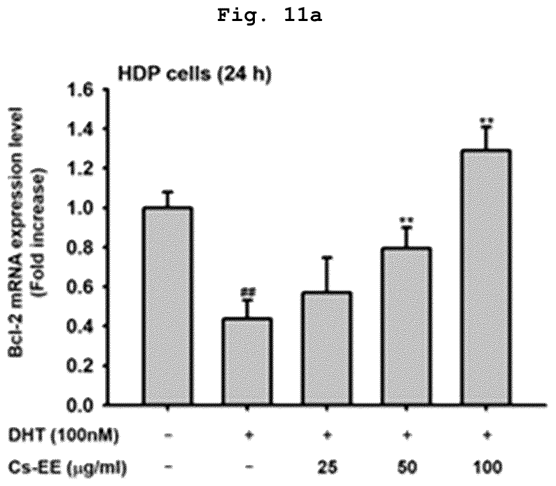

A mechanism of anti-androgen activity of Cs-EE according to the present disclosure was confirmed through 5-α reductase activity analysis. As a result, as shown in FIG. 10, when Cs-EE of 100 μg/ml or higher was treated, the 5-α reductase activity was reduced to less than 40%. Accordingly, it was confirmed that the AR protein level reduction effect of Cs-EE according to the present disclosure confirmed above was induced from the 5-α reductase activity inhibition effect of Cs-EE.

In addition, in order to confirm the hair loss prevention effect of Cs-EE in vitro, HDP cells were treated with 100 nM DHT after Cs-EE treatment, and then changes in gene mRNA expression were confirmed.

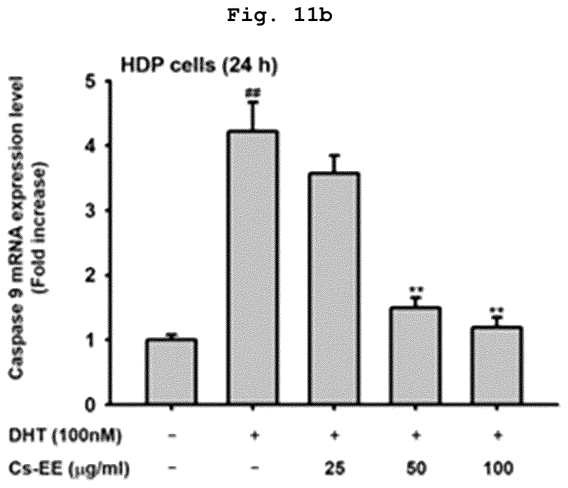

As a result, as shown in FIG. 11A, the Cs-EE of the present disclosure dose-dependently increased the Bcl-2 mRNA expression, which was reduced by DHT.

On the other hand, as shown in FIG. 11B, the Cs-EE of the present disclosure significantly reduced the expression of caspase-9 mRNA, which was a gene with pro-apoptotic properties.

Overall, the present disclosure relates to a Connarus semidecandrus Jack. extract having a hair loss treatment effect. According to the present disclosure, it has been identified that when a testosterone-induced alopecia animal model was treated with the Connarus semidecandrus Jack. extract, hair growth was promoted, hair length and thickness were also increased, the expression of androgen receptors and apoptosis-related factors was inhibited, the survival and proliferation of human dermal papilla cells were promoted, and the activity of 5-α reductase was inhibited. That is, the Connarus semidecandrus Jack. extract according to the present disclosure may be effectively and variously used for preventing or treating hair loss and promoting hair growth or hair regrowth.

1. A composition for preventing, alleviating, or treating hair loss, or for promoting hair growth or hair regrowth, the composition comprising a Connarus semidecandrus Jack. extract.

2. The composition of claim 1, wherein the extract is extracted with any one solvent selected from the group consisting of water, C1 to C4 alcohols, and mixed solvents thereof.

3. The composition of claim 1, wherein the extract is extracted from at least one part selected from the group consisting of the whole plant, aerial part, roots, leaves, seeds, and flower of Connarus semidecandrus Jack.

4. The composition of claim 1, wherein the hair loss is at least one selected from the group consisting of male pattern alopecia, female pattern alopecia, alopecia areata, and telogen effluvium.

5. The composition of claim 1, wherein the composition is a pharmaceutical composition, a quasi-drug composition, a cosmetic composition, a food composition, or a health functional food composition.

6.-13. (canceled)

14. A method for treating hair loss comprising treating a subject with a Connarus semidecandrus Jack. extract.

15. A method for promoting hair growth or hair regrowth comprising treating a subject with a Connarus semidecandrus Jack. extract.

16. The method of claim 15, wherein the extract is extracted with any one solvent selected from the group consisting of water, C1 to C4 alcohols, and mixed solvents thereof.

17. The method of claim 15, wherein the extract is extracted from at least one part selected from the group consisting of the whole plant, aerial part, roots, leaves, seeds, and flower of Connarus semidecandrus Jack.

18. The method of claim 15, wherein the hair loss is at least one selected from the group consisting of male pattern alopecia, female pattern alopecia, alopecia areata, and telogen effluvium.

19. The method of claim 14, wherein the extract is extracted with any one solvent selected from the group consisting of water, C1 to C4 alcohols, and mixed solvents thereof.

20. The method of claim 14, wherein the extract is extracted from at least one part selected from the group consisting of the whole plant, aerial part, roots, leaves, seeds, and flower of Connarus semidecandrus Jack.

21. The method of claim 14, wherein the hair loss is at least one selected from the group consisting of male pattern alopecia, female pattern alopecia, alopecia areata, and telogen effluvium.


SEQUENCE LISTING (14 sequences)

SEQ ID NO: 1 - DNA, 22 bp, synthetic construct
  source 1..22: mol_type = other DNA
atcgccctgtggatgactgagt

SEQ ID NO: 2 - DNA, 23 bp, synthetic construct
  source 1..23: mol_type = other DNA
gccaggagaaatcaaacagaggc

SEQ ID NO: 3 - DNA, 20 bp, synthetic construct
  source 1..20: mol_type = other DNA
aggcaagcagcaaagttgtc

SEQ ID NO: 4 - DNA, 22 bp, synthetic construct
  source 1..22: mol_type = other DNA
gtctttctgctcgacatcacca

SEQ ID NO: 5 - DNA, 20 bp, synthetic construct
  source 1..20: mol_type = other DNA
gacagtcagccgcatcttct

SEQ ID NO: 6 - DNA, 19 bp, synthetic construct
  source 1..19: mol_type = other DNA
gcgccaatacgaccaaatc

SEQ ID NO: 7 - DNA, 20 bp, synthetic construct
  source 1..20: mol_type = other DNA
gagtacctgaaccggcatct

SEQ ID NO: 8 - DNA, 20 bp, synthetic construct
  source 1..20: mol_type = other DNA
gaaatcaaacagaggtcgca

SEQ ID NO: 9 - DNA, 20 bp, synthetic construct
  source 1..20: mol_type = other DNA
gaaccatcatgggctggaca

SEQ ID NO: 10 - DNA, 19 bp, synthetic construct
  source 1..19: mol_type = other DNA
ggagaggaggccttcccag

SEQ ID NO: 11 - DNA, 20 bp, synthetic construct
  source 1..20: mol_type = other DNA
cgagaactaccgcaggaagc

SEQ ID NO: 12 - DNA, 20 bp, synthetic construct
  source 1..20: mol_type = other DNA
ctgtcgtattcccgcgatcc

SEQ ID NO: 13 - DNA, 20 bp, unidentified
  source 1..20: mol_type = other DNA
tgtgaacggatttggccgta

SEQ ID NO: 14 - DNA, 20 bp, synthetic construct
  source 1..20: mol_type = other DNA
actgtgccgttgaatttgcc